(19) United States
(12) Patent Application Publication
(10) Pub. No.: US 2025/0209344 A1
(43) Pub. Date: Jun. 26, 2025

(54) SYSTEM AND METHOD OF MODEL-BASED MACHINE LEARNING FOR NON-EPISODIC STATE SPACE EXPLORATION

(71) Applicant: The Research Foundation for The State University of New York, Albany, NY (US)

(72) Inventors: Josue NASSAR, Brooklyn, NY (US); Yuan ZHAO, Short Hills, NJ (US); Ian JORDAN, South Setauket, NY (US); Il Memming PARK, Stony Brook, NY (US)

(21) Appl. No.: 18/849,879

(22) PCT Filed: Apr. 4, 2023
(86) PCT No.: PCT/US2023/017378

§ 371 Date: Sep. 23, 2024

Related U.S. Application Data
(60) Provisional application No. 63/327,934, filed on Apr. 6, 2022.

(51) Int. Cl. G06N 3/0985

(52) U.S. Cl. CPC G06N 3/0985

(57) ABSTRACT

A system and method of conducting non-episodic state space exploration of a world, the world being a real-world system having one or more dimensions, including: receiving a current state of an agent observed in relation to its interaction with the world; updating one or more parameters of a dynamic model that approximates the world based on the current state of the agent, wherein the parameters as updated improve approximation of the world; generating an intrinsic reward associated with the exploration by the agent based on a closest distance of the current state of the agent in relation to a previous state of one or more previous states of the agent in its exploration of the world; and generating a control from a sequence of actions based on the dynamic model to maximize the intrinsic reward, wherein execution of the control perturbs the current state of the agent in its exploration of the world.

### CROSS-REFERENCE TO RELATED APPLICATIONS

This application is the U.S. National Phase of PCT/US2023/017378, filed on Apr. 4, 2023, and claims the benefit of U.S. Provisional Patent Application No. 63/327,934, filed on Apr. 6, 2022, the disclosures of which are incorporated by reference herein in their entireties for all purposes.

### STATEMENT OF GOVERNMENT RIGHTS

This invention was made with government support under grant no. 1845836 award by the National Science Foundation. The government has certain rights in the invention.

### BACKGROUND

### Field

The present application relates generally to model-based machine learning. More specifically, the present application is directed to a system and a method of model-based machine learning for non-episodic state space exploration.

### Brief Discussion of Related Art

Autonomous exploration of an environment about which nothing or little might be known a priori (i.e., unknown environment) aims to drive an artificial agent to efficiently learn about the environment through active investigation of that environment. Applications of such exploration include neuroscience, robotics, and artificial intelligence, among many other applications.

The ability to autonomously explore the unknown environment is critical to the artificial agent's success. Exploration expands the horizon of knowledge, leads to discovery of rewards and dangers, opens new potential actions, and allows for better-informed behavior. Autonomous exploration is a notoriously difficult task for an artificial agent and has largely remained an open problem. Reinforcement learning is a subset of machine learning. While many solutions have been proposed in reinforcement learning literature, where autonomous exploration is necessary to maximize the agent's performance, these solutions face two main limitations that prevent them from being deployed in many realistic environments.

The first limitation stems from allowing an arbitrary number of resets to complete exploration. It is convenient and popular to model the exploration problem as episodic, such that after each episode (i.e., experimental trial) of exploration in the unknown environment, the artificial agent “respawns” at a (potentially new) location in the environment. The respawning allows for multiple identical copies of the agent to simultaneously transverse the environment, resulting in parallel training when possible. While such a formulation is suitable for simulated environments, where direct access to a simulator is available (e.g., video games), these conditions are not feasible in many real-world scenarios (e.g., a rover on the surface of Mars, or within a context of personalized medicine). In such real-word scenarios, episodic exploration is infeasible as it might not be possible to respawn or restart the artificial agent at a selected location to continue exploration. In short, the agent cannot be freely placed or duplicated in such real-world scenarios.

For example, in a neurological application where a neuro-stimulator is interfaced with a patient's brain to alleviate pathological symptoms via electrical current stimulation, the artificial agent (i.e., representing a current state of the brain or a pattern of its electrical activity), as controlled by the neuro-stimulator, may need to explore internal patterns of neural activity of the patient's unique brain with injuries and/or illnesses that are specific to that patient. However, the internal state of the patient's brain cannot be arbitrarily reset, thus preventing the artificial agent from starting over. As such, complete exploration of the patient's brain may not be possible in this paradigm.

The second limitation stems from a Markovian reward common to reinforcement learning systems, where a probability of a chosen action (e.g., stimulation in the neurological application) and therefore also the next state of the artificial agent are not history dependent on artificial agent's previous states, but only take into account the current state of the artificial agent. In the foregoing regard, the reward, which is crucial to deriving the majority of reinforcement learning systems, is Markovian (static) reward that does not allow the artificial agent to keep track of where it has explored and where it has not explored, as the reward does not change with respect to the agents traversal of the environment from its initialization.

While reinforcement learning systems thrive in various simulated environments, the problem of non-episodic exploration of real-word environments remains elusive. It is therefore desirable to provide a system and method of model-based machine learning for non-episodic state space exploration that includes a dynamic model of the environment which keeps track of the explored parts and yet to be explored parts, along with a non-Markovian (dynamic) reward that incentivizes the artificial agent to keep exploring unknown, yet to be explored parts of the unknown environment, as calculated and maximized based on previously explored parts of the environment.

### SUMMARY

There are provided a system and a method of model-based machine learning for non-episodic state space exploration.

In accordance with an embodiment or aspect, there is disclosed a method of conducting non-episodic state space exploration of a world, the world being a real-world system having one or more dimensions, wherein the method includes: receiving a current state of an agent observed in relation to its interaction with the world; updating one or more parameters of a dynamic model that approximates the world based on the current state of the agent, wherein the parameters as updated improve approximation of the world; generating an intrinsic reward associated with the exploration by the agent based on a closest distance of the current state of the agent in relation to a previous state of one or more previous states of the agent in its exploration of the world; and generating a control from a sequence of actions based on the dynamic model to maximize the intrinsic reward, wherein execution of the control perturbs the current state of the agent in its exploration of the world.

In some cases, receiving the current state can include sensing one or more signals in relation to the interaction of the agent with the world using one or more sensors, while in other cases receiving the current state can further include estimating the current state from the one or more signals as sensed.

The method can further include generating the dynamic model. In some cases, generation of the dynamic model can include providing one or more hyper-parameters that define a structure of the dynamic model, and initializing the one or more parameters of the dynamic model that provide an initialized approximation of the world for the exploration by the agent.

In some cases, the closest distance is one of a Euclidian distance, Euclidian distance squared, L1 distance, L-infinity distance, cosine distance, Chebyshev distance, Jaccard distance, Haversine distance, Sørensen-Dice distance, Manhattan distance, Minkowski distance, Hamming distance, Mahalnobis distance, or another type of distance metric.

The method can further include generating a new landmark for the current state if the closest distance to a center of a landmark associated with the previous state is greater than or equal to a predetermined distance, and updating a counter of a previous landmark associated with the previous state if a center of the previous landmark is the closest distance from the current state and the closest distance is less than the predetermined distance.

In some cases, generating the intrinsic reward associated with the exploration by the agent is based on the closest distance of the current state of the agent in relation to a landmark associated with a previous state of one or more previous states of the agent in its exploration of the world.

The method can further include generating the sequence of actions based on the dynamic model that maximizes the intrinsic reward, and selecting a first action from the sequence of actions as the control. In some cases the generation of the sequence of actions that maximizes the intrinsic reward can include: generating a plurality of sequences, each of the plurality of sequences including an associated number of actions capable of resulting in possible future states of the agent; generating intrinsic rewards associated with the possible future states in each of the plurality of sequences; summing the intrinsic rewards to generate a total reward for each of the plurality of sequences; and selecting one of the plurality of sequences that has a highest total reward as the sequence of actions that maximizes the intrinsic reward.

The method can further include executing the control to perturb the current state of the agent in its exploration of the world.

In accordance with another embodiment or aspect, there is disclosed a system to conduct non-episodic state space exploration of a world, the world being a real-world system having one or more dimensions. The system includes a computing device, and a memory device storing instructions that, when executed by the computing device, cause the computing device to perform the following operations.

The operations of the system include: receiving a current state of an agent observed in relation to its interaction with the world; updating one or more parameters of a dynamic model that approximates the world based on the current state of the agent, wherein the parameters as updated improve approximation of the world; generating an intrinsic reward associated with the exploration by the agent based on a closest distance of the current state of the agent in relation to a previous state of one or more previous states of the agent in its exploration of the world; and generating a control from a sequence of actions based on the dynamic model to maximize the intrinsic reward, wherein execution of the control perturbs the current state of the agent in its exploration of the world.

In some cases, operations associated with receiving the current state can include sensing one or more signals in relation to the interaction of the agent with the world using one or more sensors, while in other cases receiving the current state can further include estimating the current state from the one or more signals as sensed.

The operations of the system can further include generating the dynamic model. In some cases, operations associated with generating the dynamic model can include providing one or more hyper-parameters that define a structure of the dynamic model, and initializing the one or more parameters of the dynamic model that provide an initialized approximation of the world for the exploration by the agent.

In some cases, the closest distance is one of a Euclidian distance, Euclidian distance squared, L1 distance, L-infinity distance, cosine distance, Chebyshev distance, Jaccard distance, Haversine distance, Sørensen-Dice distance, Manhattan distance, Minkowski distance, Hamming distance, Mahalnobis distance, or another type of distance metric.

The operations of the system can further include generating a new landmark for the current state if the closest distance to a center of a landmark associated with the previous state is greater than or equal to a predetermined distance, and updating a counter of a previous landmark associated with the previous state if a center of the previous landmark is the closest distance from the current state and the closest distance is less than the predetermined distance.

In some cases, generating the intrinsic reward associated with the exploration by the agent is based on the closest distance of the current state of the agent in relation to a landmark associated with a previous state of one or more previous states of the agent in its exploration of the world.

The operations of the system can further include generating the sequence of actions based on the dynamic model that maximizes the intrinsic reward, and selecting a first action from the sequence of actions as the control. In some cases, operations associated with generation of the sequence of actions that maximizes the intrinsic reward can include: generating a plurality of sequences, each of the plurality of sequences including an associated number of actions capable of resulting in possible future states of the agent; generating intrinsic rewards associated with the possible future states in each of the plurality of sequences; summing the intrinsic rewards to generate a total reward for each of the plurality of sequences; and selecting one of the plurality of sequences that has a highest total reward as the sequence of actions that maximizes the intrinsic reward.

The operations of the system can further include executing the control to perturb the current state of the agent in its exploration of the world.

These and other purposes, goals and advantages of the present application will become apparent from the following detailed description of example embodiments read in connection with the accompanying drawings.

### BRIEF DESCRIPTION OF THE DRAWINGS

Some embodiments are illustrated by way of example and not limitation in the figures of the accompanying drawings in which:

FIG. 1 illustrates a graphical representation of an example world that exhibits two attractors and an agent that conducts exploration of the world based on certain actions that advance or move the agent in the world across a plurality of time steps resulting in a plurality of states of the agent;

FIG. 2 illustrates a graphical representation of another example world that exhibits an agent that conducts exploration of the example world based on certain actions that advance or move the agent in the world across a plurality of time steps resulting in a plurality of states of the agent;

FIG. 3 illustrates a block diagram of a dynamic model that approximates a world;

FIG. 4 illustrates a block diagram of an example world and a non-episodic state space exploration system capable of conducting exploration of the example world based on certain actions executed by an action executor that advances or moves the agent in the world across a plurality of time steps resulting in a plurality of states of the agent, observed by sensors and used to refine a dynamic model of the world as it is explored;

FIG. 5A illustrates generation of a new landmark and initialization of an associated counter for a state of the agent based on one or more landmarks associated with previous states of the agent within the world;

FIG. 5B illustrates increase of a landmark's counter associated with a state of the agent that is within a predetermined distance of one or more previous states of the agent within the world;

FIG. 6 illustrates a block diagram of an example neurological system in which an example world represent neural activity of a person's brain and a non-episodic state space exploration system capable of conducting state space exploration of the example world based on certain actions (controls) of an agent executed across a plurality of time steps resulting in a plurality of states of the agent, observed and used to refine and/or update a dynamic model of the world as it is explored;

FIGS. 7A-7F illustrate several example representations of how the non-episodic state space exploration system in accordance with FIGS. 4 and 6 refines and/or updates the dynamic model that approximates the world, as the agent explores the world over a plurality of time steps resulting in a plurality of states of the agent;

FIG. 7G illustrates an example of the world showing flow dynamics and two attractors, approximated by the example representations in accordance with FIGS. 7A-7F;

FIG. 8 illustrates a flowchart of an example method of conducting non-episodic state space exploration using an artificial agent that explores an unknown world (unknown environment) based on a dynamic model of the world and an intrinsic reward maximized based on previously explored parts of the world; and

FIG. 9 illustrates a block diagram of an example general computer system including instructions capable of execution that causes the system to perform methods and/or computer-based functions described herein and illustrated with reference to FIGS. 1-8.

### DETAILED DESCRIPTION

A system and method associated with model-based machine learning for non-episodic state space exploration are disclosed herein. In the following description, for the purposes of explanation, numerous specific details are set forth to provide a thorough understanding of example embodiments or aspects. It will be evident, however, to one skilled in the art, that an example embodiment may be practiced without all the disclosed specific details.

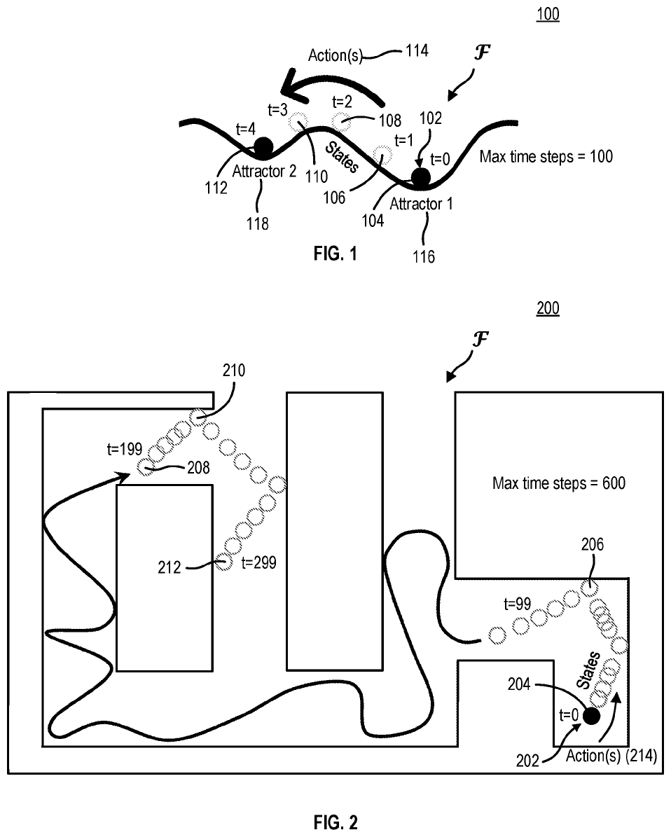

FIG. 1 illustrates a graphical representation of an example world [symbol] (100) that exhibits two attractors 116, 118 and an agent 102 that conducts exploration of the world 100 based on certain actions 114 executed by the agent 102 across a plurality of time steps t resulting in a plurality of states 104-112 of the agent 102.

In general, a state (e.g., any one of the states 104-112) is a set of numerical quantities that describes a position (location) of the agent 102 in the world 100, while a world (e.g., world 100) is a collection of all combinations of the numerical quantities obtainable. For example, if one were to model the state of a ball being thrown through in a three-dimensional world (e.g., our world), a state would be 3-dimensional, i.e., three (3) numbers that explain the ball's position in x-y-z space.

In general, it should be noted that a particular world could be a single-dimensional world or a multidimensional world, e.g., as illustrated in FIG. 1.

In particular, the world 100 is an example two-dimensional representation (e.g., x-y space) of a hypothetical peak/valley world instructive to assist with understanding of non-episodic state space exploration described herein (e.g., hereinafter referred to as “state space exploration” or simply “exploration”). It should be noted that real worlds can be and are infinitely more complex, i.e., n-dimensional worlds where n could be tens to hundreds of dimensions in practical applications and could be theoretically upwards of billions of dimensions in the case of the human brain. The system and method described herein are applicable to the exploration of such worlds, assuming there exist capabilities that can generate relatively low-dimensional representations of such worlds on the order of tens to several hundred dimensions, to maintain computational feasibility.

The agent 102 is an artificial construct that explores the world 100 through active exploration, as instructed by actions 114. It should be noted that actions 114 can be used synonymously with the terms controls or perturbations. For the example peak/valley world 100, the agent 102 can be considered a ball 102. As the ball 102 is rolled, its location (state) changes in the peak/valley world (state space) 100 over time steps t. As already desired hereinabove, applications of such exploration include neuroscience, robotics, and artificial intelligence, among many other applications. In the various applications, the agent 102 is synonymous with a state of the agent 102, as the position (location) of the agent 102 in the world 100 is its state by definition.

Attractors 116, 118 are states 104, 112 which attract the agent 102 because one or more small actions (also known as perturbations) 114 may insufficiently push a current state 104, 112 of the agent 102 away from the attractors 116, 118 in state space, but at a later time step t the agent 102 will revert or fall back to such states 104, 112. As an example, if the agent 102 (ball) is pushed insufficiently from the attractor 116, it will revert to or fall back to the attractor 116 at a later time step t. In this regard, it should be noted that through execution of one or more actions 114, the agent 102 can overcome the attraction of the attractor 116 and the state of the agent 102 can thus transition from the attractor 116 to the attractor 118. It should be noted that the attractors 116, 118 can also be considered states.

For example, the agent 102 positioned at state 104 (at time step t=0) and attracted to attractor 116 can transition to state 112 (at time step t=4) and be attracted to another attractor 118 through the application of several actions 114 resulting in intermediate states 106, 108, and 110 at respective times t=1, t=2, and t=3.

As an example, the maximum number of time steps for the exploration of example world 100 could be set to one hundred steps (or another maximum number) and/or a terminal condition can be defined (e.g., terminal state), after meeting the earlier of which exploration of the world 100 can be ceased or stopped. It should be noted that the maximum number of time steps is settable and/or selectable and can be dependent on the complexity of the world to be explored, and/or dependent on other dependencies such as medical requirements in neurological applications or monetary requirements in financial applications. Similarly, a terminal condition can be defined, such as for example, when a certain attractor is reached (e.g., attractor 118), or a number of attractors is reached that were explored in neurological, robotic, chemical, or financial applications, among others.

FIG. 2 illustrates a graphical representation of another example world [symbol] (200) that exhibits an agent 202 that conducts exploration of the example world 200 based on certain actions 214 executed by the agent 202 across a plurality of time steps t resulting in a plurality of states 204-212 of the agent 202.

As aforementioned, it should be noted that a particular world could be a single-dimensional world or a multidimensional world, e.g., as illustrated in FIG. 2.

In particular, the world 200 is an example two-dimensional representation of a hypothetical maze world to further assist with understanding of non-episodic state space exploration described herein. The agent 202 is an artificial construct that explores the world 200 through active exploration, as instructed by the actions 214. For example, in maze world 200, the agent 202 can be also considered a ball 202. As the ball rolls, its location (state) changes in the maze (state space). Unlike the peak/valley world 100, which had two attractors 116, 118, any state of the ball 202 in the maze world 200 is considered an attractor.

A simple way to consider the difference is that in the world 200 the agent 202 (ball) is attracted at every state it visits 204-212 during exploration at respective time steps t=0 through t=299, whereas in world 100 the agent 102 (ball) will revert to the attractors 116, 118 unless sufficiently perturbed. Another simple way to consider this difference is that in the world 200 if the agent 202 (ball) is picked up and dropped it will be attracted to a state where it lands, whereas in the world 100 the agent 102 (ball) will revert back to the attractors 116, 118, i.e., fall back or roll to the attractors 116, 118.

It should be noted that states 204-212 are labeled for simplicity and for clarity, and it should be understood that there can be a multiplicity of states at times t=0 to t=299. In the maze world 200, any action 214 executed by the agent 202, will perturb the agent from one state into another state along the world 200.

As an example, a maximum number of time steps for the exploration of example world 200 could be set to six hundred steps (or another maximum number) and/or a terminal condition can be defined, after meeting the earlier of which exploration of the world 200 can be ceased or stopped. It should be noted that the maximum number of time steps is settable and/or selectable, and can be dependent on the complexity of the world to be explored, and/or dependent on other dependencies as aforementioned. Similarly, a terminal condition can be defined, such as for example, when a certain attractor is reached or a number of explored attractors is reached as aforementioned.

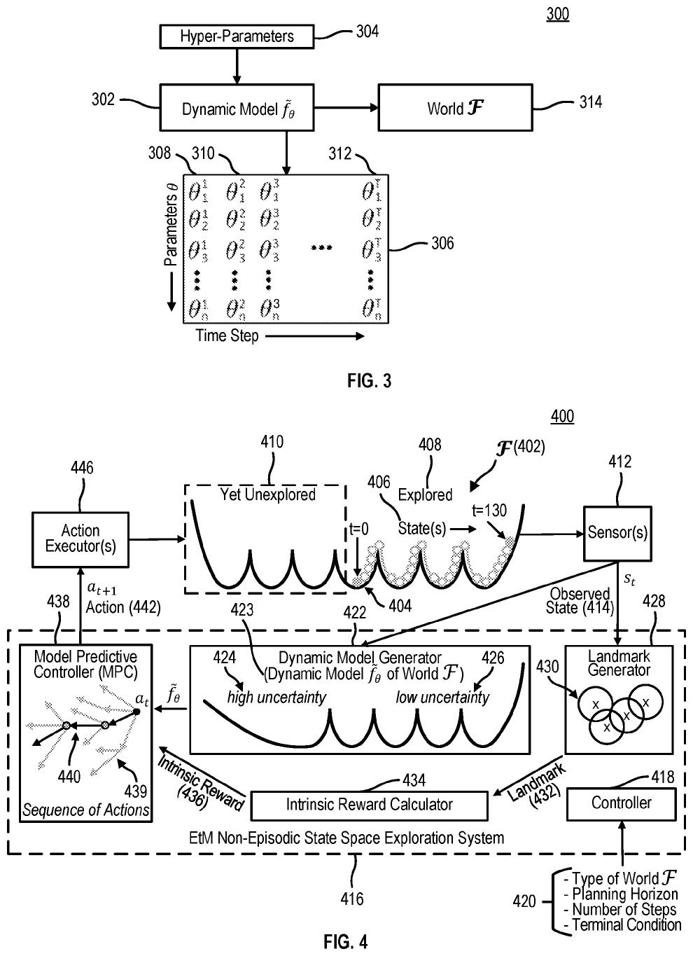

It should be noted that while the hypothetical worlds 100, 200 are illustrated in FIGS. 1 and 2 for clarity and brevity of the description, these worlds 100, 200 are often unknown and cannot be easily visualized as they are explored (traversed) by the agents 102, 202, but worlds 100, 200 can be approximated by respective models as described in greater detail below with reference to FIG. 3.

FIG. 3 illustrates a block diagram 300 of a dynamic model 302 that approximates a world [symbol] 314. In real-world applications, the world 314 is unknown and is initially modeled as a dynamic model \tilde{f}_{θ} 302 which is then refined as an agent (e.g., agents 102, 202) explores the particular world 314 (e.g., worlds 100, 200).

The dynamic model 302 is a probabilistic model that approximates the world 314. It should be noted that the world 314 could be neural, biological, ecological, physical, chemical, mechanical, fluid, genetic, cryptographical, financial, or another world [symbol] that can be described, expressed, and/or modeled using the dynamic model 302. Some example probabilistic models that can be used include artificial neural networks, linear dynamical systems, Gaussian processes, and/or other probabilistic models. In general, the dynamic model 314 is refined (updated) iteratively to maximize a probability of seeing a current state of an agent, given a last action taken along with the agent's previous state.

The dynamic model 302 includes hyper-parameters 304 that constrain the dynamic model of the world 314 and how its parameters θ are updated, and a matrix 306 or a set 308, 310, . . . ,312 of parameters θ that are updated iteratively in real-time as the agent (e.g., 102, 202) explores the world 314 in order to approximate the world 314 over a number of time steps.

The hyper-parameters 304 generally constrain the dynamic model \tilde{f}_{θ} 314 of the particular world [symbol] 314, defining its structure and learning capabilities, e.g., different hyper-parameters would be chosen for the different example worlds 100, 200. Depending on the probabilistic model used for the dynamic model 302 of world 314, a set of hyper-parameters is thus selected to define that probabilistic model of the dynamic model 302. For example, if an artificial neural network is used to model the world [symbol] 314, a user would identify attributes of the neural network that might include a number of hidden layers (e.g., layers of neurons), a width of each layer (e.g., how many neurons exist in each layer), a learning rate, etcetera. These attributes would then be used to define hyper-parameters 304 that constrain the dynamic model 302 of the world 314.

The matrix 306 includes one or more sets 308, 310, . . . , 312 of parameters 0. Each set of parameters corresponds to a time step as the agent explores the world 314. In particular, once the hyper-parameters have been defined for the dynamic model 302, the dynamic model 302 will then include one or more sets of parameters 308, 310, . . . 312, with each set including θ₁ through θₙ parameters, which alter or refine the dynamic model's 302 approximation of the world 314 because these parameters change with the agent's exploration of the world 314 (e.g., exploration by agent 102, 202 of the respective world 100, 200), over each of the time steps t up to the maximum time steps T, wherein t=1, 2, 3, . . . ,T. At each time step t, the θ parameters are adjusted to better approximate the world 314 based on the state of agent as it interacts with the world 314. It should be noted that the sets 308, 310, . . . , 312 of parameters θ can be saved at each time step for later evaluation, or in the alternative, the first set 308 can be overwritten with parameters θ associated with each successive time step as the agent explores (traverses) the world 314.

It should be noted that the parameters θ depend on the probabilistic model used to approximate the world 314 and not on the world 314 itself. For example, if a neural network were used to approximate the world 314, the parameters θ would represent synaptic strengths (e.g., connections between neurons). Prior to the agent beginning exploration of the world 314, the parameters θ of the dynamic model will be set (e.g., usually randomly), with each element sampled from a Gaussian or uniform distribution. However, more sophisticated initialization strategies can of course be used, e.g., orthogonal initialization, Xavier initialization, normalized Xavier initialization, and He initialization, among other initialization strategies, depending on a cellular level architecture (e.g. a hyper-parameter) of the neural network.

FIG. 4 illustrates a block diagram 400 of an example world [symbol] 402 and a non-episodic state space exploration system 416 capable of conducting exploration of the example world 402 based on certain actions executed by an action executor(s) 446 that advances or moves (perturbs) the agent 404 in the world 402 across a plurality of time steps t resulting in a plurality of states 406 of the agent, observed by sensor(s) 412 and used to refine a dynamic model 423 of the world 402 as it is explored.

The non-episodic state space exploration system 416 includes a controller 418, a dynamic model generator 422, a landmark generator 428, an intrinsic reward calculator 434, and a model-based predictive controller (MPC) 438.

The controller 418 is configured to receive information 420 related to non-episodic state space exploration, including a selection of a type of the world [symbol] 402 for exploration, a planning horizon H associated with a sequence of actions to be predicted at each time step, a maximum number of time steps T for conducting the exploration, and a terminal condition in connection with the exploration of the world 402.

Moreover, the controller 418 is configured to communicate with the dynamic model generator 422 to generate (instantiate) and initialize a dynamic model \tilde{f}_{θ} 423 for the type of the world 422 selected in connection with the state space exploration. Lastly, the controller 418 controls iterative exploration and termination conducted by the system 416 over the indicated maximum number of time steps T (e.g., time step t<max time steps T) and/or whether the terminal condition is reached (e.g., a current state has reached a certain attractor state, a certain number of attractors has been reached, or a predefined limit for certain actions has been reached (e.g., a number of actions executed in a certain direction, such as 150 moves of the agent 404 to the right, the left, or another direction)).

The dynamic model generator 422 is configured to generate and/or initialize a dynamic model \tilde{f}_{θ} 423 for the type of the world 422 selected in connection with the state space exploration. The world 402 is an example peak-valley world [symbol] approximated by the dynamic model 423, which is an example of the dynamic model 302 as described hereinabove with reference to FIG. 3. In particular, the dynamic model 423 generated by the dynamic model generator 422 includes hyper-parameters that model the structure and learning constraints of the dynamic model 423 for the world 402, and a set or a matrix of parameters θ (e.g., set 308 or matrix 306 as illustrated in FIG. 3). The dynamic model generator 422 is configured to initialize and then update interactively in real-time the parameters θ as the agent 404 explores the world 402, in order to refine or better approximate the world 402 over the time steps t.

Concerning initialization of the dynamic model 423, the hyper-parameters that describe the structure and the constraints of the dynamic model 423 could be automatically associated with the selected type of the world 402 and parameters θ could be automatically initialized when a user selects the type of world [symbol] communicated to the dynamic model generator 422, or the foregoing hyper-parameters and parameters θ could be initially entered and/or modified by the user via the controller 418 and then saved for the current and/or future explorations of the type of world [symbol] .

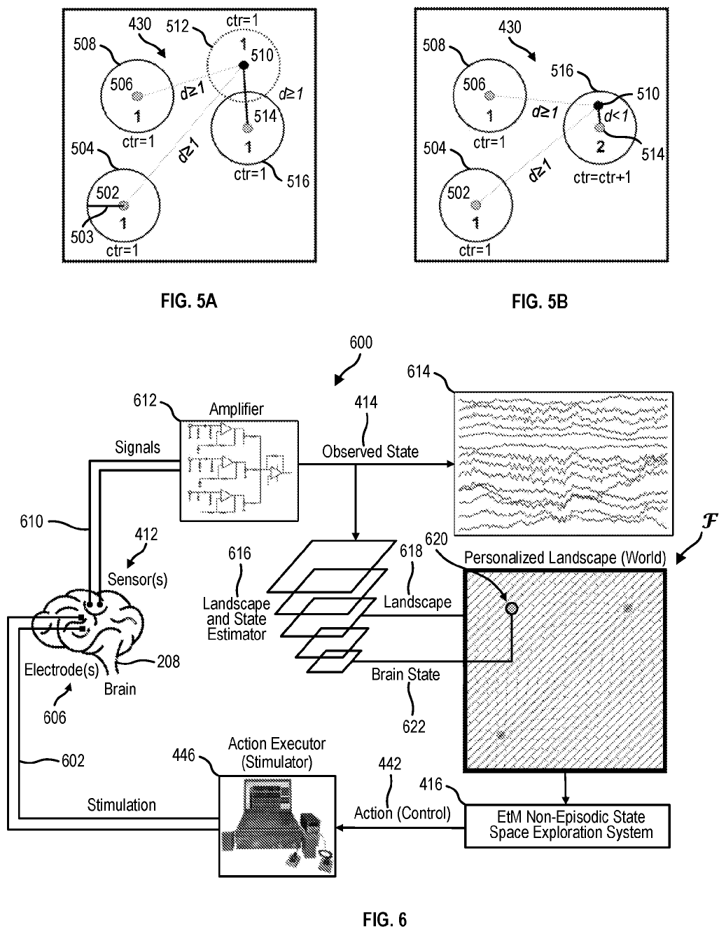

The landmark generator 428 is configured to receive an observed (current) state 414 of the agent 404 in the world 402 from a sensor(s) 414, and based on the observed state 414, the landmark generator 428 is further configured to either generate a new landmark or update a counter of a previously generated landmark, which are hereinafter referenced to as landmarks 430. More specifically, the landmark generator 428 is configured to generate a new landmark about a center of the observed state 414 if there are no previously generated landmarks that are within a predetermined distance d of the observed state 414 of the agent 404, or further configured to increment a counter of an existing and previously generated landmark that is closest to the observed state 414 of the agent 404 and within the predetermined distance d. The landmark generator 428 is configured to maintain and update the landmarks 430 as the agent 404 explores the world 402. Generation and update of the landmarks 430, as well as associated determinations, will be described in greater detail hereinbelow with reference to FIGS. 5A and 5B.

In some example embodiments not employing landmarks 430, the intrinsic reward calculator 434 is configured to generate an intrinsic (non-Markovian) reward 436 based on the following reward function:

$$i ( {s}_{t} ; {S}_{t} ) := {min}_{j < t} d ( {s}_{t} , {s}_{j} ) ,$$

wherem Sₜ is an observed (current) state of the agent, Sₜ is a set of all states previously visited by the agent, i (sₜ; Sₜ) is a reward function based on the current state among all states, and

$${min}_{j < t} d ( {s}_{t} , {s}_{j} )$$

is a value of a distance from the current state to a closest state of all previously visited states (i.e., minimum distance).

In other example embodiments employing landmarks 430, the intrinsic reward calculator 434 is configured to receive or access the landmark 432 generated or updated for the current state as well as all landmarks 430 previously generated and/or updated for states that the agent 404 has previously visited, and further configured to generate an intrinsic (non-Markovian) reward 436, which is a value based on the following reward function:

$$i ( {s}_{t} ; {S}_{t} ) = 𝕝 [ {s}_{t} ∈ {B}_{k} ] \frac{1}{{N}_{k}} + 𝕝 [ {s}_{t} ∉ {B}_{k} ] d ( {s}_{t} , {s}_{k} )$$

wherein
sₜ: is an observed (current) state of the agent;
Sₜ: is a set of all states previously visited by the agent;
i(sₜ; Sₜ): is a reward function based on the current state among all states previously visited;
Bₖ: one landmark of k-many landmarks;
Nₖ: counter of how many times the landmark was visited;
d(sₜ, sₖ): distance from the current state to a closest state of all states previously visited

$$𝕝 [ {s}_{t} ∈ {B}_{k} ] \frac{1}{{N}_{k}} :$$

if the current state is within one of k-many landmarks, then the reward function i(sₜ; Sₜ) returns a value of

$$\frac{1}{{N}_{k}} ;$$

and
[symbol] [sₜ∉Bₖ]d (sₜ, sₖ): otherwise, if the current state is not within one of k-many landmarks, then the reward function i(sₜ; Sₜ) returns a value of a distance from the current state to the closest state of all states previously visited.

To incentivize the agent 404 to explore the world 402, the intrinsic reward 436 depends on both the current state of the agent 404 and all previous states that the agent 404 has visited (agent's state history), as represented by the landmarks 430 that consolidate the agent's state history, wherein such consolidation significantly reduces the computational and memory requirements. The intrinsic reward 436 is calculated such that a higher reward is given the farther away the agent 404 moves from any of the previously visited states 406, as represented by the landmarks 430. This reward 436 is intrinsic to the agent 404 (e.g., internal to the agent), as no external reward from the world 402 is present or given as a reward for the agent's moves. Moreover, the dependence of the intrinsic reward 436 on all previous states 406 that the agent 404 has visited in the world 402 makes this intrinsic reward also inherently non-Markovian. Accordingly, the reward 436 is both intrinsic and non-Markovian.

As described hereinabove, St is a set of all states previously visited by the agent, and is an ever-growing set as the agent 404 explores or traverses the state space of the world 402. As further described above, the distance d(.,.) represents a distance from a current state to a closest state of all states 406 that the agent 404 previously visited. It should be noted that any type of distance metric can be used for the distance between the states, such Euclidian distance (e.g., Euclidian distance squared), L1 distance, L-infinity distance, cosine distance, Chebyshev distance, Jaccard distance, Haversine distance, Sørensen-Dice distance, Manhattan distance, Minkowski distance, Hamming distance, Mahalnobis distance, or another type of distance metric that is now known or developed in the future in connection with machine learning in various systems and/or implementations. In some embodiments, a Euclidian distance squared is used as the type of distance metric. It should be noted that a distance metric does not have to strictly follow a formal mathematical definition for a distance metric, e.g., one that obeys a triangular inequality, and can thus include any metric that can be used to indicate distances, such as a Euclidean distance, a Euclidean distance squared, etcetera.

In operation, the agent explores the world 402 in a certain direction (e.g., traversing all the valleys to the left or the right side (in this case the right) of its starting state at time step t=0), wherein the intrinsic reward 436 is calculated and maximized (increased) to incentivize the agent 404 to explore the world 402. After the intrinsic reward 436 is calculated and sufficiently low in the direction of exploration (e.g., about time step t=130), such that sequences of actions 440 favor distant regions of the opposite and yet unexplored part 410 of the world 402, the agent 404 will be motivated to backtrack based on the intrinsic reward related to all previously visited states in conjunction with the MPC 438 (discussed hereinbelow in greater detail), allowing the agent 404 to complete the exploration of the state space of the world 402. For example, after the state space exploration moves to the extreme right state at time step t=130, the next state would be a previously-visited state and so on until the agent 404 backtracks through all the states it has previously visited to get back to the initial state at time step t=0. After this initial state, the agent 404 will then continue to the left to explore the yet unexplored part 410 of the world 402.

The MPC 438 is configured to receive or access the dynamic model \tilde{f}_{θ} 423 generated by the dynamic model generator 422 and receive or access the intrinsic reward 436 calculated by the intrinsic reward calculator 434. Based on planning horizon H received or accessed from the controller 418, the dynamic model 423, and the intrinsic reward 436, the MPC 438 is further configured to generate a sequence of actions at (controls) 440 at a time step t that maximizes the intrinsic reward of the agent 404 so that it continues to explore toward high uncertainty 424 of the dynamic model 422.

In view of the foregoing, if the agent 404 moves inside a predetermined distance from a landmark 432, then the agent 404 is given a reward of less than one (e.g., calculated intrinsic reward<1). However, if the agent 404 moves outside of the predetermined distance from any of the existing landmarks 430, a new landmark is generated and the agent 404 is given a reward of greater than one (e.g., calculated intrinsic reward>1). The MPC 438 then determines or predicts a sequence of future actions 440 that maximize the calculated intrinsic reward over time steps t up to the planning horizon H. These actions can move the agent 404 closer to or farther away from the existing landmarks 430. In the case of backtracking through the states of the previously explored part 408 to reach the yet unexplored part 410, MPC 438 may find that at each time step t a maximum reward can be achieved by traveling near the previously visited landmarks 430. However, this might be very situationally dependent. In practice, the movement of the agent 404 will depend on the given world 402, the state of the agent 404 with respect to the world 402, and all the previous landmarks 430 that are employed in calculating the intrinsic reward 436 and determining the sequence of actions 404 maximizing the intrinsic reward 436.

In order to predict a sequence of actions (controls) 440 that maximizes the intrinsic reward 436 over the dynamic model 423, the MPC 438 computes or predicts a plurality of random sequences of future actions 439, wherein each predicted sequence includes a set of actions that result in expected states ranging from the current state Sₜ 414 at time step t through to a state at the planning horizon H, e.g., a state S_{t+H} at time step t+H. Each action in each of the random sequences is drawn from a uniform random distribution or a custom distribution that incorporates known properties of the world 402 as reflected by the dynamic model 423, such as geometry and topology of local and global dynamic features about the world 402. For example, if the world is of a form of the example peak/valley world 100 and is monotonic (e.g., always increasing in one direction), the distribution can be biased by which actions are sampled in order to more often chose those actions that perturb the agent up the hill so as to compensate for gravity (or analogous features) attracting the agent to the bottom of the hill.

In each of the random sequences, an individual intrinsic reward 436 is computed for each state that is expected as a result of a random action, using the intrinsic reward calculator 434. The MPC 438 then sums the intrinsic rewards to generate a total intrinsic reward for each of the random sequences. A random sequence with a highest total intrinsic reward (maximized intrinsic reward) is then chosen as the sequence of actions 440, e.g., sequence of actions αₜ for time steps t through t+H. It should be noted that the number of random sequences 439 that are generated can range from 200 to 1500 random sequences per time step t. Greater or fewer sequences of actions can be generated.

After computing or predicting a random sequence of actions (controls) 440 that maximizes the intrinsic reward, the MPC 438 selects a first predicted action (control) αₜ₊₁ 442 from the sequence of actions (controls) 440. Thereafter, the action executor 446 executes the action (control) αₜ₊₁ 442 to advance or move (perturb) the agent 404 from the observed (current) state 414 to a next state that explores or traverses the state space of the world 402.

In view of the foregoing and as particularly illustrated in FIG. 4, the system 416 is capable of conducting state space exploration of the world 402 based on actions (controls) executed by an action executor(s) 446 that advances or moves the agent 404 (e.g., ball) in the world 402 across time steps t resulting in states sₜ 406 of the agent 404, observed by sensor(s) 412 and used to update or refine a dynamic model 423 of the world 402 as it is explored, so that the model 423 more closely or accurately approximates the world 402. As further illustrated, the agent 404 has explored only a part 408 of the world 402 across for example 130 time steps, but still has to explore the yet unexplored part 410 of the world 402. For the explored part 408 of the world 402, the parameters θ of the dynamic model 423 reflect low uncertainly 426 as to the model's approximation of the state space of the world 402. However, for the yet unexplored part 410 of the world 402, the parameters θ of the dynamic model 423 reflect high uncertainly 424 as to the model's approximation of the state space of the world 402, and reflect a generally uninformed approximation or understanding of the yet unexplored part 410 of the world 402. This approximation may be close to the initial approximation, but not necessarily the exact initial representation. For example, the approximation may be close to the initial approximation by chance, but not necessarily the exact initial representation, as refining and/or updating the parameters of the dynamic model 423 amounts to changes across the entire approximation of the world 402.

In operation of the system 416, the sensor(s) 412 will determine the observed (current) state sₜ of the agent 404 in the world 402. Based on the observed state sₜ, the landmark generator 428 generates either a new landmark 432 about a center of the state sₜ (e.g., with a radius r=1) if there are no previously generated landmarks that are within a predetermined distance d (e.g., d≥1) of the observed state 414 of the agent 404, or increments the counter of an existing and previously generated landmark 432 that is closest to the current state st among previously generated or updated landmarks 430 and within the predetermined distance d (e.g., d<1). The intrinsic reward calculator 434 uses the foregoing landmark information to generate an intrinsic reward 436. Contemporaneously therewith, dynamic model generator 422 uses the observed (current) state to update the dynamic model 423 of the world 422, and thus over time reduces uncertainty related to the dynamic model 422 from high uncertainty to low uncertainty for the new states of the agent within the world 402.

The MPC 428 uses the dynamic model 423 generated and updated/refined by the dynamic model generator 422 and the intrinsic reward 436 calculated by the intrinsic reward calculator 434, to generate a sequence of actions (controls) 440 that are expected to maximize the intrinsic reward 436, as described in greater detail hereinabove. The first action (control) aₜ 442 of the sequence actions (controls) 440 is executed by the executor 446 to advance or move (perturb) the state of the agent in the world 402 from the observed current state 414 to a next state at a further time step t.

The evolution of the agent's current state can be expressed with the following formula:

$${s}_{t} = f ( {s}_{t - 1} , {a}_{t} ) + ε$$

wherein
sₜ: current state of the agent at a time step t;
f(.,.): dynamics expressed by the world [symbol] ;
sₜ₋₁: previous state of the agent at a time step t−1;
αₜ: action (control) executed at a given time step t; and
ε: random noise drawn from some distribution at each time step t.

FIG. 5A illustrates generation of an example new landmark 432 and initialization of an associated counter s_{t,lm} for a state sₜ of the agent 404 based on one or more landmarks 430 associated with previous states of the agent 404 within the world 402.

As particularly illustrated in FIG. 5A, landmarks 504, 508, and 516 have been generated and respectively associated with states 502, 506, and 514 of the agent 404 (e.g., sₜ, wherein t=0, 1,2 . . . , N), maintained as the landmarks 430 by landmark generator 428 as illustrated in FIG. 4. Each of the landmarks 504, 508, and 516 is generated as a circle having a radius 503 equal to a predetermined distance, such as a distance of one (e.g., radius r=1) about a center of the associated state sₜ. Moreover, other radius values can be used (e.g., r=1.5). In similar fashion, other shapes or combinations of shapes, as well as their states' defined sizing, can of course be used instead of the circle and its radius. It should be noted that any type of distance metric can be used for the distance between the states, such Euclidian distance (e.g., Euclidian distance squared), L1 distance, L-infinity distance, cosine distance, Chebyshev distance, Jaccard distance, Haversine distance, Sørensen-Dice distance, Manhattan distance, Minkowski distance, Hamming distance, Mahalnobis distance, or another type of distance metric that is now known or developed in the future in connection with machine learning in various systems and/or implementations. In some implementations, a Euclidian distance squared is used as the type of distance metric. As aforementioned, a distance metric does not have to strictly follow a formal mathematical definition for a distance metric, e.g., one that obeys a triangular inequality, and can thus include any metric that can be used to indicate distances, such as a Euclidean distance, a Euclidean distance squared, etcetera.

When each of the landmarks 504, 508, and 516 is initially generated, its associated counter is initialized to one (e.g., s_{t,lm}=1). The counter indicates a number of times that the agent 404 has visited within the predetermined distance of a particular previous state sₜ, and more particularly, a landmark associated with the particular previous state sₜ. As illustrated, the agent 404 has visited each of the landmarks 504, 508, and 516 only once. Subsequent visitation of a state or landmark associated with the state, and the associated increase of the state's counter will be described hereinafter with reference to FIG. 5B.

For a new landmark to be generated, the distance d from an observed (current) state sₜ 414 to a closest previous state, and more particularly, a center of a closest landmark of any previous state has to be greater than or equal to the predetermined distance (e.g., d≥1). As the distances d between centers of example landmarks 504, 508, and 516 are greater than or equal to the predetermined distance, these landmarks were newly generated as the agent 404 visited each of the associated states 502, 506, 514, and they are maintained as landmarks 430 with associated counters initialized to one.

When the agent 404 visits an observed current state sₜ 510, the distance d from the current state 510 to a closest previous state 514, and more particularly, the center of the closest landmark 516 of any previous state 514 has to be greater than or equal to predetermined distance (e.g., d≥1) for the creation of a new landmark. The distance d from the center of landmark 516, associated with state 514, is closest to the observed current state st 510 among landmarks 430 maintained by the landmark generator 428 (e.g., landmarks 504, 508, 514). Because the distance d is greater than or equal to predetermined distance (e.g., d≥1), the landmark generator 428 generates a new landmark 512 about the center of the state 510, and the landmark's counter is initialized to one (e.g., s_{t,lm}=1)

FIG. 5B illustrates increase of a landmark 432 counter Stim associated with a state sₜ of the agent 404 that is within a predetermined distance d of one or more previous states 406 of the agent 404 within the world 402.

In contrast to FIG. 5A, when the agent 404 visits an observed current state st 510 as illustrated in FIG. 5B, the distance d from the current state 510 to a closest previous state 514, and more particularly, a center of the closest landmark 516 of any previous state 514 is less than the predetermined distance (e.g., d<1). As before, the distance d from the center of landmark 516, associated with state 514, is closest to the observed current state st 510 among landmarks 430 maintained by the landmark generator 428 (e.g., landmarks 504, 508, 514). Because the distance d is less than the predetermined distance (e.g., d<1), the landmark generator 428 does not add a new landmark but rather increments the counter of the closest landmark 516 (e.g., s_{t,lm}=1). Accordingly, the counter of the landmark 516 associated with state 514 now equals to two (e.g., s_{t,lm}=2).

FIG. 6 illustrates a block diagram of an example neurological system 600 in which the world 618 represents neural activity of a person's brain 608 and a non-episodic state space exploration system 416 capable of conducting state space exploration of the example world 618 based on certain actions (controls) 442 of an agent 620 executed across a plurality of time steps t resulting in a plurality of states of the agent 620, observed and used to update and/or refine a dynamic model of the world 618 as it is explored.

The neurological system 600 includes non-episodic state space exploration system 416, action executor (neuro-stimulator) 446, electrodes 606, sensors 412, amplifier 612, and landscape and state estimator 616.

The non-episodic state space exploration system 416 that is applicable to neurological system 600 has been described in detail hereinabove with reference to FIG. 4. The dynamic model used by the system 416 in the neurological system 600 includes the structure and function as described with reference dynamic models 302, 422 of FIGS. 3 and 4. In particular, hyper-parameters 304 are selected to model the neurological structure of the world 618 for a person's brain 608, and parameters θ are initialized and then updated interactively in real-time as the agent 620 explores the state space of the world 618, in order to refine or better approximate the world 618 over the time steps t.

The action executor 446 receives the action (control) 442 and executes the action 442, e.g., providing stimulation (perturbation) 602 to the brain 608 via one or more electrodes 606. The stimulation can be electrical stimulation (e.g., intracranial stimulation, cranial stimulation, transcranial magnetic stimulation, or other remote brain stimulation), light to the retinas, touch (e.g., mechanical or electrical stimulation to the body), smell/odors to the nose, taste to the tongue, other types of stimulation, and/or one or more combinations intended to move the state of the agent 620 from one state to another state.

Using intracranial electrical stimulation as an example, electrodes 606 can be implanted in various parts of the brain 608. Depending on the type of neurological condition being investigated, the one or more electrodes 606 can be implanted in various parts of the brain 608 or can be surface electrodes. In general, the electrodes 606 can be placed anywhere on the central or peripheral nervous system. This includes, scalp, surface of the brain, inside the brain, or on a nerve (e.g., vagus nerve stimulation). Other forms of stimulation need to take into account sensory processes that they target (e.g., light to the retinas, touch to somewhere on the body, smell to the nose, taste to the tongue, etc).

The sensors 412 alone or in combination with an amplifier 612 sense one or more signals from the brain 608 and provide the observed state 414 to the non-episodic state space exploration system 416. The observed state 414 can be one or more signal traces 614 as provided by the sensors 412. In neurological applications, a multiplicity of sensors (e.g., 32, 64, or 385 sensors) can be used to provide the observed state 414 of the brain 608. In some cases, the landscape and state estimator 616 can generate a low-dimensional representation of the world 618 and the observed state 622, e.g., reducing the dimensions of the multiplicity of signals resulting from the sensors 412. The non-episodic state space exploration system 416 can use observed state 622 instead of the observed state 414 of the world 618.

In operation, the non-episodic state space exploration system 416 acquires an observed (current) state 414 from the sensors 412, or observed (current) state 622 from the landscape and state estimator 616. Based on current state 414 or 622, the dynamic model generator 422 updates its dynamic model 423 of the world 618 and the landmark generator generates a new landmark or updates an existing landmark 432 from its maintained landmarks 430. The MPC 438 generates a sequence of actions (controls) 440 and transmits a first action 442 of the sequence 440 to the action executor 446. The action executor 446 receives the action (control) 442 and executes this action (control) 442, e.g., providing stimulation 602 to the brain 608 via one or more electrodes 606, wherein the stimulation 602 can be a specific pattern of stimulation having an amplitude and a frequency. Stimulation patterns are generally determined a priori by a type of neuro-stimulator used (e.g., a common one is biphasic current injection neuro-stimulator). In a simplest case, stimulation of whatever type and/or pattern is simply applied or not applied. More complex patterns of stimulation could include and vary an intensity of stimulation, as well as a modulation of the frequency of stimulation, or an adjustment of the “duty cycle” in the biphasic case.

The sensors 412 obtain the next observed state 414 of the brain 608 or the next observed state 622 is generated by the landscape and state estimator 616 such that the next observed state 414 or 622 is inputted to the non-episodic state space exploration system 416, which repeats the processing described hereinabove for the next observed state 414. The non-episodic state space exploration system 416 continues the state space exploration until a maximum of the time steps T is reached or a certain terminal condition is reached.

The agent 620, as controlled by the action executor 446, is capable of exploring internal patterns of neural activity (states) of the patient's unique brain 608 that might have injuries and/or illnesses that are specific to that patient, such as where the patient might be in a coma or vegetative state. During the agent's state space exploration, a pathological state can be observed, where further exploration of a distance attractor state (e.g., representing a terminal condition) that treats (e.g., alleviates or cures) the patient's pathological state or associated symptoms may exist (e.g., transitioning from a coma state to a wakeful state.)

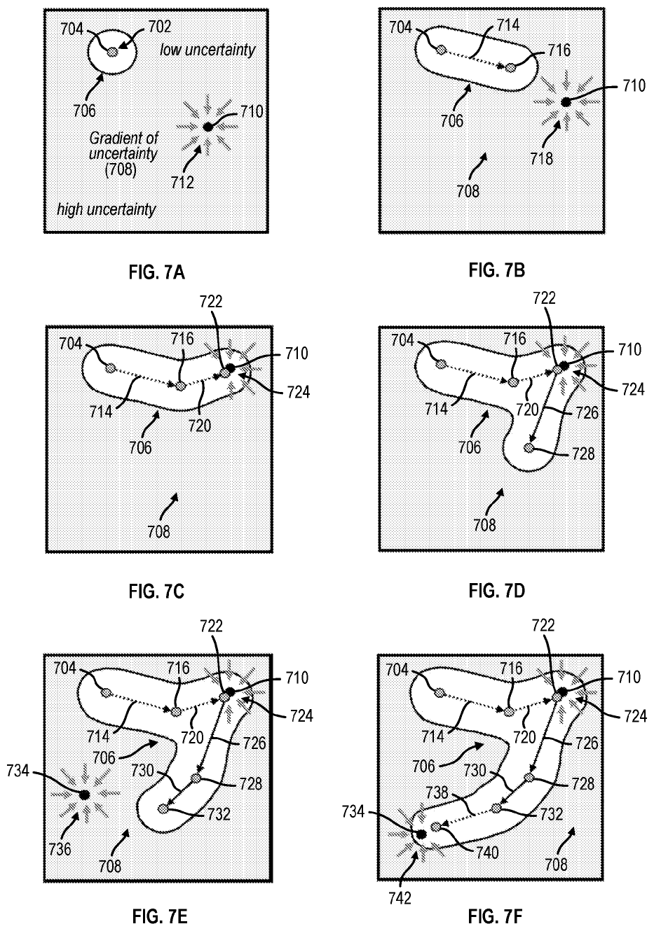

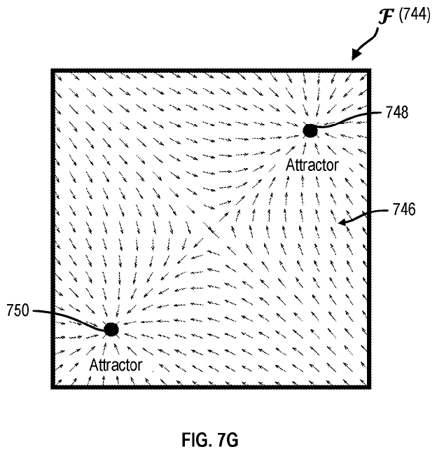

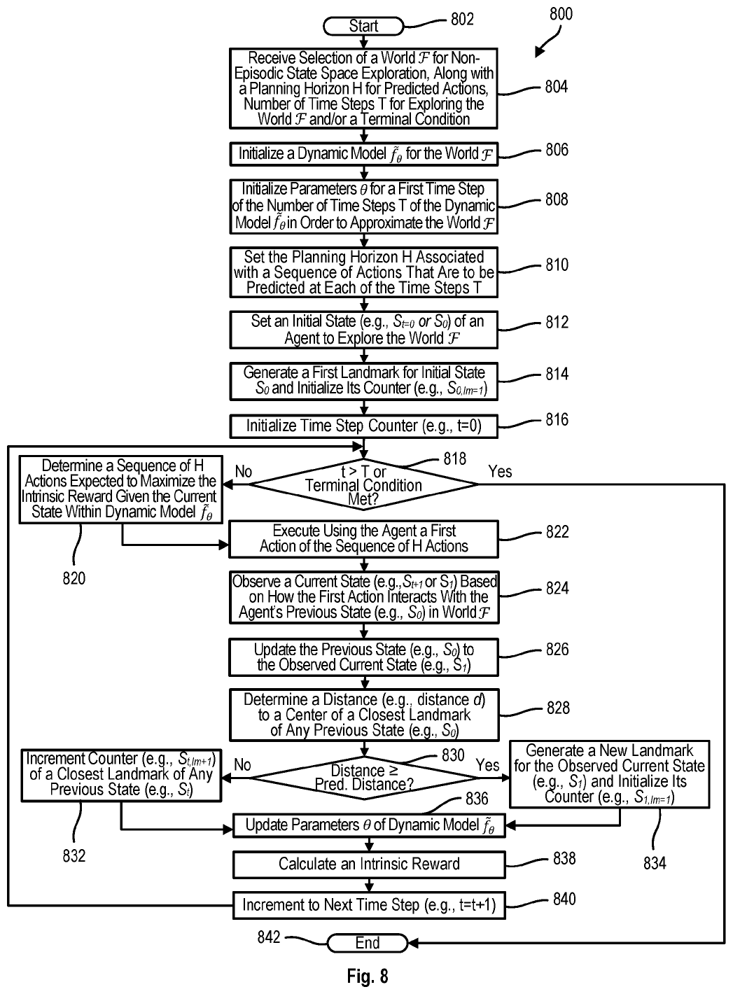

FIGS. 7A-7F illustrate several example representations of how the non-episodic state space exploration system 416 in accordance with FIGS. 4 and 6 refines and/or updates the dynamic model \tilde{f}_{θ} that approximates the world [symbol] 618, as the agent explores the state space of the world over a plurality of time steps t resulting in a plurality of states of the agent.

As illustrated in FIG. 7A, a current state 704 of an agent 702 represents neural dynamics expressed by world 618 and approximated in the dynamic model \tilde{f}_{θ} at a time step t (e.g., t=0). In particular, the dynamic model \tilde{f}_{θ} indicates that there is low uncertainty 706 of the state space of the world [symbol] 618 about the current state 704 of the agent 702. However, as the remaining state space of the world 618 is yet to be explored, the dynamic model fe indicates there is high uncertainty farther away from the current state 704 of the agent 702. More specifically, there is a continuous gradient of uncertainty 708 over the entire approximation of the world 618 (e.g., shaded in gray). It should be noted that the binary representation between the low uncertainty close to the explored state 704 and the high uncertainty farther away from the explored state 704 is simply for clarity purposes.

Moreover, based on the dynamics as expressed by world 618 and approximated by dynamic model \tilde{f}_{θ} at the state 704 of the agent 702, a first attractor 710 can be inferred from the available knowledge of the state space and the neural dynamics converging at an estimated location 712. As the agent 702 is far away, the existence of the first attractor is inferred from the neural dynamics, but the location of the attractor 710 is not known exactly and is thus crudely estimated.

As illustrated in FIG. 7B, autonomous neural dynamics represented by a dashed line 714 (e.g., not resulting from an action 442 executed by action executor 446) result in state 716 expressed by world 618 and approximated in the dynamic model \tilde{f}_{θ} at a next time step t (e.g., t=1). Over the several time steps t, the dynamic model \tilde{f}_{θ} has been updated or refined and now indicates that there is low uncertainty 706 of the state space of the world [symbol] 618 about states 704, 716 of the agent 702. As the agent 702 is now closer to the inferred first attractor 710, the location of the first attractor 710 is refined and better estimated as location 718 in the dynamic model \tilde{f}_{θ}.

As further illustrated in FIG. 7C, autonomous neural dynamics represented by a dashed line 720 result in state 722 expressed by world 618 and approximated in the dynamic model \tilde{f}_{θ} at a next time step t (e.g., t=2). Over the several time step t, the dynamic model \tilde{f}_{θ} has been updated or refined and now indicates that there is low uncertainty 706 of the state space of the world [symbol] 618 about states 704, 716, 722 of the agent 702. As the agent 702 is now closer to the inferred attractor 710, the location of the first attractor 710 is refined and estimated as location 724 in the dynamic model \tilde{f}_{θ}.

As is clearly illustrated in FIGS. 7A-7C, the location of the inferred first attractor 710 has been updated or refined several times to the latest location 724 as the agent 702 explored the state space of the world 618 over time steps t (e.g., from t=0 to (=2) based on autonomous neural dynamics expressed by world 618.

As illustrated in FIG. 7D, neural dynamics represented by a solid line 726 are a result of an action 442 executed by action executor 446, resulting in state 728 expressed by world 618 and approximated in the dynamic model \tilde{f}_{θ} at a next time step t (e.g., t=3). Over the past several time steps t, the dynamic model \tilde{f}_{θ} has been updated and/or refined and now indicates that there is low uncertainty 706 of the state space of the world [symbol] 618 about states 704, 716, 722, 728 of the agent 702.

As further illustrated in FIG. 7E, neural dynamics represented by a solid line 730 are a result of another action 442 executed by the action executor 446, resulting in state 732 expressed by world 618 and approximated in the dynamic model \tilde{f}_{θ} at a next time step t (e.g., t=4). Over the past several time steps t, the dynamic model fo has been updated or refined and now indicates that there is low uncertainty 706 of the state space of the world [symbol] 618 about states 704, 716, 722, 728, 732 of the agent 702.

Moreover, based on neural dynamics expressed by world 618 and approximated by dynamic model \tilde{f}_{θ} at state 732 of the agent 702, a second attractor 734 can now be inferred from the available knowledge of the state space and the neural dynamics converging at an estimated location 736. As the agent 702 is far away, the existence of the second attractor is inferred from the neural dynamics, but the location 736 of the second attractor 734 not known exactly and is crudely estimated.

As illustrated in FIG. 7F, autonomous neural dynamics represented by a dashed line 738 (e.g., not resulting from an action 442 executed by action executor 446) result in state 740 expressed by world 618 and approximated in the dynamic model \tilde{f}_{θ} at a next time step t (e.g., t=5). Over the several time steps t, the dynamic model \tilde{f}_{θ} has been updated or refined and now indicates that there is low uncertainty 706 of the state space of the world [symbol] 618 about states 704, 716, 722, 728, 732, 740 of the agent 702. As the agent 702 is now closer to the inferred second attractor 734, the location of the second attractor 734 is refined and better estimated as location 742 in the dynamic model \tilde{f}_{θ}.

As is illustrated in FIGS. 7A-7F, the states of the agent 702 can result from autonomous neural dynamics alone, or neural dynamics resulting from a perturbation over the autonomous dynamics via an action 442 executed by an action executor 446. As the agent 702 explores the state space of the world 618 over time steps t (e.g., t=0 to t=5), the dynamic model \tilde{f}_{θ} is updated or refined to better approximate the world 618, with the uncertainty being reduced as the agent explores from regions of low uncertainty towards regions of high uncertainty, as directed by maximization of the intrinsic reward 436 based on the dynamic model \tilde{f}_{θ} and landmarks 430 of previously visited states.

In the example FIGS. 7A-7F, the first attractor 710 can represent an example attractor state (e.g., coma state), while the second attractor 736 can represent another example attractor state (e.g., wakeful state). Of course, state space exploration and associated treatment therapies will depend on specific global dynamics brought about by a disorder being targeted, as well as the cognitive and/or behavioral consequences of the brain state existing in any given attractor state at a given time step t.

FIG. 7G illustrates an example of the world [symbol] 744 showing flow dynamics 746 and two attractors 748, 750, approximated by the example representations of a dynamic model \tilde{f}_{θ} in accordance with FIGS. 7A-7F.

Using the non-episodic state space exploration system 416 in conjunction with system 600, a dynamic model \tilde{f}_{θ} of world 744 can be initialized and updated/refined as the agent 620 explores the state space of the world 618 over time steps t up to maximum time steps T or a terminal condition, to better approximate or converge the dynamic model \tilde{f}_{θ} to the world 744.

FIG. 8 illustrates a flowchart of an example method 800 of conducting non-episodic state space exploration using an artificial agent that explores an unknown world (unknown environment) based on a dynamic model of the world and an intrinsic reward maximized based on previously explored parts of the world.

The example method 800 starts at operation 802, wherein the non-episodic space exploration system 416 is set up and configured such that it can interact with the unknown world [symbol] using one or more sensors 412 that provide an observed (current) state 414, and one or more action executors 446 that can execute an action 442 such that an agent 404 can explore the unknown world [symbol] . The particular sensors and action executors that are utilized depend on the nature of the world being considered, e.g., control of a rover on Mars versus control of a state of a person's brain, etc.

At operation 804, there is received a selection of an unknown world [symbol] for non-episodic state space exploration, along with a planning horizon H for predicted actions, a maximum number of time steps T for exploring the world and a terminal condition associated with ceasing or stopping the exploration.

At operation 806, a dynamic model \tilde{f}_{θ} of the world [symbol] is initialized. As already described hereinabove, the dynamic model \tilde{f}_{θ} includes hyper-parameters describing the structure of the world and a set of parameters θ that approximate the world [symbol] , wherein the parameters θ are updated or refined as the agent explores the world's state space.

At operation 808, initialize parameters θ for a first time step of the maximum number of time steps T of the dynamic model \tilde{f}_{θ} in order to approximate the world [symbol] . At operation 810, set the planning horizon H associated with a sequence of actions that are to be predicted at each of the time steps t of the maximum time Steps T.

At operation 812, an initial state of an agent (e.g., Sₜ₌₀ or S₀) is set in order to explore the world [symbol] . The setting of the initial state can be a result of observation via sensors 412, and in some cases, further estimation via the landscape and state estimator 616. At operation 814, generate a first landmark for the initial state S₀ and initialize its counter (e.g., S_{0,lm}=1). At operation 816, initialize time step counter (e.g., t=0).

Thereafter, at operation 818 a determination is made as to whether the time step t is greater than the maximum number of time steps T (e.g., t>T), or whether the terminal condition has been met (e.g., current state has reached a certain attractor state).

If it is determined at step 818 that the maximum time steps or the terminal condition has been reached, the method continues at operation 842 where the method 800 ends or terminates. However, if it is determined at step 818 that the maximum time steps or the terminal condition has not been reached, the method continues at operation 820, where a sequence of H actions expected to maximize an intrinsic reward given a current (or initial) state within the dynamic model \tilde{f}_{θ} is determined. Where the current state is the initial state (e.g., S₀), a default intrinsic reward of one (e.g., intrinsic reward=1) can be used or can be calculated in relation to the first and only state and the first and only landmark generated for the initial state, as respectively generated in operations 812 and 814. For example, the intrinsic reward i(sₜ; Sₜ) described with reference to FIG. 4 would be calculated as follows:

$$i ( {s}_{t} ; {S}_{t} ) = 𝕝 [ {s}_{t} ∈ {B}_{k} ] \frac{1}{{N}_{k}} = \frac{1}{1} = 1.$$

At operation 822, execute using the agent a first action of the sequence of H actions. At operation 824, a next state (e.g., Sₜ₊₁ or S₁) of the agent is observed based on how the first action interacts with the agent's current state (e.g., S₀) in the in the world world [symbol] . At operation 826, the current state (e.g., S₀) is updated to the observed next state (e.g., Sₜ). At operation 828, a distance (e.g., distance d) is determined to a center of a closest landmark for any previous state (e.g., S₀).

At operation 830, a determination is made as to whether a distance d from a center of the observed next state to a center of a closest landmark for any previous state is greater or equal to a predetermined distance (e.g., d≥1).

If it is determined at operation 830 that the distance is greater than or equal to the predetermined distance (e.g., d≥1), then at operation 834 a new landmark is generated for the observed next state (e.g., S₁) and the associated counter is initialized to one (e.g., S_{1,lm=1}). However, if it is determined at operation 830 that the distance is less than the predetermined distance (e.g., d<1), then at operation 832 the counter for the closest landmark of any previous state (e.g., Sₜ) is incremented by one (e.g., S_{t,lm+1}).

Thereafter, at operation 836 the parameters θ of the dynamic model \tilde{f}_{θ} are updated to refine or better approximate the world [symbol] . At operation 838, an intrinsic reward is calculated. At operation 840, the time step t is incremented to the next time step (e.g., t=t+1). Thereafter, the example method 800 continues at operation 818 and can iterate operations 820-840 until the maximum of time steps T is reached or the terminal condition is reached at operation 818.

In view of the description in FIGS. 1-8, a system and a method of model-based machine learning for non-episodic state space exploration have been described. In embodiments described herein, the system and method can intelligently monitor, explore, and navigate ways that various systems behave, such as a neural, biological, chemical, mechanical, fluid, genetic, financial, or another system. In various “physical” applications (e.g., biological, chemical, mechanical, fluid, genetic, financial), the system and method can serve as means for system identification of multi-stable worlds, that is, determining how the agent will respond to various input perturbations (e.g., determining the input/output relationship) when the agent is in one of a variety of different achievable attractor states. The field of system identification is crucial for the testing and safety protocols of many engineering projects regardless of the discipline.

In neural applications, the system and method can be used to treat brain disorders, ranging from movement impairments and comas, to psychiatric disorders, such as depression and obsessive compulsive disorder (OCD). The system and method enable exploration and mapping of brain states, corresponding to various behaviors, as well as cognitive functions and/or dysfunctions. As such, the space of achievable neural states, in a given context, can be explored, navigated, and treated. This has applications to both the research community, for understanding how underlying neural dynamics bring rise to the necessary computations to enact function and/or behavior, and the medical community, as such, enabling transition of a patient out of brain states, associated with dysfunctions, such as comas.

The system and method enable exploration and mapping a person's internal brain states in a desired context, achievable through various stimulation techniques, including neuro-stimulation (e.g., electrical pulses), transcranial currents, optogenetic manipulation, auditory stimulus, visual stimulus, and other stimulation techniques. This system and method is a paradigm change from currently available systems, enabling more thorough and complete exploration of the states of the person's brain. This system and method can in real-time induce transitions out of a stable state (e.g., coma), by observing neural activity, processing the observed activity to continuously build an internal dynamic model of the person's neural system, and delivering stimulation in a desired form based on the updated and/or refined dynamic model. While useful in a research setting, as a means to more efficiently analyze the neural dynamics, the system and method have various immediate clinical applications as well.

For example, patients suffering from disorders of consciousness, such as a coma, may benefit from such a stimulation, as the system and method can facilitate a transition from the stable neural state giving rise to the dysfunctional behavior (e.g., coma) into another neural stable state related to better functional behavior (e.g., wakefulness). As another example, in Parkinson's disease, the system and method can provide stimulation that relieves a patient of unwanted symptoms (e.g., unintended or uncontrollable movements, such as shaking, stiffness, and difficulty with balance and coordination).

The system and method provide a solution to treating and/or better understanding multiple neurological dysfunctions, including, but not limited to, movement disorders, epilepsy, and disorders of consciousness.

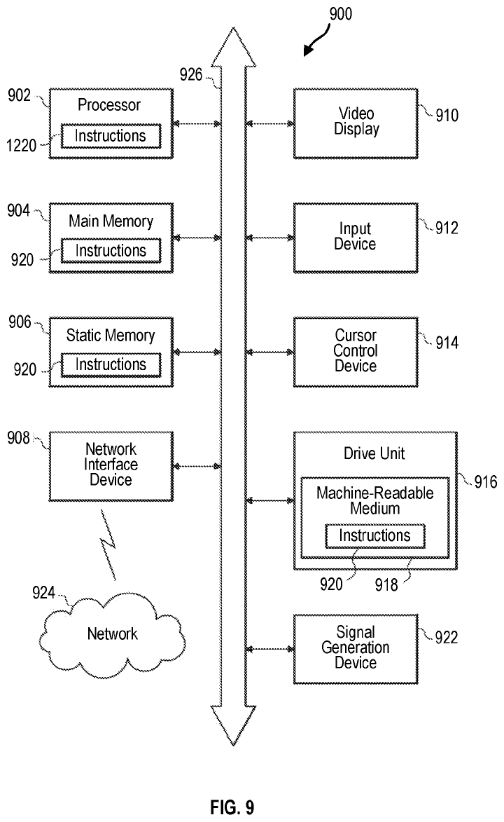

FIG. 9 illustrates a block diagram of an example general computer system 900. The computer system 900 can include a set of instructions that can be executed to cause the computer system 900 to perform any one or more of the methods or computer-based functions described herein and illustrated with reference to FIGS. 1-8. The computer system 900, or any portion thereof, may operate as a standalone device or may be connected, e.g., using a network or other connection, to other computer systems or peripheral devices. For example, the computer system 900 may be a non-episodic state space exploration system 416, an action executor 446, and/or amplifier 612, and may further be connected to other systems and devices directly (e.g., serially) or via the network 924.

The computer system 900 may also be implemented as or incorporated into various devices, such as a personal computer (PC), a tablet PC, a personal digital assistant (PDA), a computing device or mobile device (e.g., smartphone), a palmtop computer, a laptop computer, a desktop computer, a communications device, a control system, a web appliance, or any other machine capable of executing a set of instructions (sequentially or otherwise) that specify actions to be taken by that machine. Further, while a single computer system 800 is illustrated, the term “system” shall also be taken to include any collection of systems or sub-systems that individually or jointly execute a set, or multiple sets, of instructions to perform one or more computer functions.

As illustrated in FIG. 9, the computer system 900 may include a processor 902, e.g., a central processing unit (CPU), a graphics-processing unit (GPU), or both. Moreover, the computer system 900 may include a main memory 904 and a static memory 906 that can communicate with each other via a bus 926. As shown, the computer system 900 may further include a video display unit 910, such as a liquid crystal display (LCD), an organic light emitting diode (OLED), a flat panel display, a solid state display, or a cathode ray tube (CRT). Additionally, the computer system 900 may include an input device 912, such as a keyboard, and a cursor control device 914, such as a mouse. The computer system 900 can also include a disk drive (or solid state) unit 916, a signal generation device 922, such as a speaker or remote control, and a network interface device 908.

In a particular embodiment or aspect, as depicted in FIG. 9, the disk drive (or solid state) unit 916 may include a computer-readable medium 918 in which one or more sets of instructions 920, e.g., software, can be embedded. Further, the instructions 920 may embody one or more of the methods or logic as described herein. In a particular embodiment or aspect, the instructions 920 may reside completely, or at least partially, within the main memory 904, the static memory 906, and/or within the processor 902 during execution by the computer system 900. The main memory 804 and the processor 802 also may include computer-readable media.

In an alternative embodiment or aspect, dedicated hardware implementations, such as application specific integrated circuits, programmable logic arrays and other hardware devices, can be constructed to implement one or more of the methods described herein. Applications that may include the apparatus and systems of various embodiments or aspects can broadly include a variety of electronic and computer systems. One or more embodiments or aspects described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

In accordance with various embodiments or aspects, the methods described herein may be implemented by software programs tangibly embodied in a processor-readable medium and may be executed by a processor. Further, in an exemplary, non-limited embodiment or aspect, implementations can include distributed processing, component/object distributed processing, and parallel processing. Alternatively, virtual computer system processing can be constructed to implement one or more of the methods or functionality as described herein.

It is also contemplated that a computer-readable medium includes instructions 920 or receives and executes instructions 920 responsive to a propagated signal, so that a device connected to a network 924 can communicate voice, video or data over the network 924. Further, the instructions 920 may be transmitted or received over the network 924 via the network interface device 808.

While the computer-readable medium is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein.

In a particular non-limiting, example embodiment or aspect, the computer-readable medium can include a solid-state memory, such as a memory card or other package, which houses one or more non-volatile read-only memories. Further, the computer-readable medium can be a random access memory or other volatile re-writable memory. Additionally, the computer-readable medium can include a magneto-optical or optical medium, such as a disk or tapes or other storage device to capture carrier wave signals, such as a signal communicated over a transmission medium. A digital file attachment to an e-mail or other self-contained information archive or set of archives may be considered a distribution medium that is equivalent to a tangible storage medium. Accordingly, any one or more of a computer-readable medium or a distribution medium and other equivalents and successor media, in which data or instructions may be stored, are included herein.

In accordance with various embodiments or aspects, the methods described herein may be implemented as one or more software programs running on a computer processor. Dedicated hardware implementations including, but not limited to, application specific integrated circuits, programmable logic arrays, and other hardware devices can likewise be constructed to implement the methods described herein. Furthermore, alternative software implementations including, but not limited to, distributed processing or component/object distributed processing, parallel processing, or virtual machine processing can also be constructed to implement the methods described herein.

It should also be noted that software that implements the disclosed methods may optionally be stored on a tangible storage medium, such as: a magnetic medium, such as a disk or tape; a magneto-optical or optical medium, such as a disk; or a solid state medium, such as a memory card or other package that houses one or more read-only (non-volatile) memories, random access memories, or other re-writable (volatile) memories. The software may also utilize a signal containing computer instructions. A digital file attachment to e-mail or other self-contained information archive or set of archives is considered a distribution medium equivalent to a tangible storage medium. Accordingly, a tangible storage medium or distribution medium as listed herein, and other equivalents and successor media, in which the software implementations herein may be stored, are included herein.

Thus, a system and a method of model-based machine learning for non-episodic state space exploration have been described. Although specific example embodiments or aspects have been described, it will be evident that various modifications and changes may be made to these embodiments or aspects without departing from the broader scope of the invention. Accordingly, the specification and drawings are to be regarded in an illustrative rather than a restrictive sense. The accompanying drawings that form a part hereof, show by way of illustration, and not of limitation, specific embodiments or aspects in which the subject matter may be practiced. The embodiments or aspects illustrated are described in sufficient detail to enable those skilled in the art to practice the teachings disclosed herein. Other embodiments or aspects may be utilized and derived therefrom, such that structural and logical substitutions and changes may be made without departing from the scope of this disclosure. This Detailed Description, therefore, is not to be taken in a limiting sense, and the scope of various embodiments or aspects is defined only by the appended claims, along with the full range of equivalents to which such claims are entitled.

Such embodiments or aspects of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments or aspects have been illustrated and described herein, it should be appreciated that any arrangement calculated to achieve the same purpose may be substituted for the specific embodiments or aspects shown. This disclosure is intended to cover any and all adaptations or variations of various embodiments or aspects. Combinations of the above embodiments or aspects, and other embodiments or aspects not specifically described herein, will be apparent to those of skill in the art upon reviewing the above description

The Abstract is provided to allow the reader to quickly ascertain the nature and gist of the technical disclosure. It is submitted with the understanding that it will not be used to interpret or limit the scope or meaning of the claims.

In the foregoing description of the embodiments or aspects, various features are grouped together in a single embodiment for the purpose of streamlining the disclosure. This method of disclosure is not to be interpreted as reflecting that the claimed embodiments or aspects have more features than are expressly recited in each claim. Rather, as the following claims reflect, inventive subject matter lies in less than all features of a single disclosed embodiment or aspect. Thus each claim stands on its own as a separate example embodiment or aspect. It is contemplated that various embodiments or aspects described herein can be combined or grouped in different combinations that are not expressly noted in the Detailed Description. Moreover, it is contemplated that claims covering such different combinations can similarly stand on their own as separate example embodiments or aspects.

1. A method of conducting non-episodic state space exploration of a world, the world being a real-world system having one or more dimensions, the method comprising:
receiving a current state of an agent observed in relation to its interaction with the world;
updating one or more parameters of a dynamic model that approximates the world based on the current state of the agent, wherein the parameters as updated improve approximation of the world;
generating an intrinsic reward associated with the exploration by the agent based on a closest distance of the current state of the agent in relation to a previous state of one or more previous states of the agent in its exploration of the world; and
generating a control from a sequence of actions based on the dynamic model to maximize the intrinsic reward, wherein execution of the control perturbs the current state of the agent in its exploration of the world.

2. The method of claim 1, wherein receiving the current state comprises sensing one or more signals in relation to the interaction of the agent with the world using one or more sensors.

3. The method of claim 2, wherein receiving the current state further comprises estimating the current state from the one or more signals as sensed.

4. The method of claim 1, further comprising generating the dynamic model.

5. The method of claim 4, wherein generation of the dynamic model comprises:
providing one or more hyper-parameters that define a structure of the dynamic model; and
initializing the one or more parameters of the dynamic model that provide an initialized approximation of the world for the exploration by the agent.

6. The method of claim 1, wherein the closest distance is one of a Euclidian distance, Euclidian distance squared, L1 distance, L-infinity distance, cosine distance, Chebyshev distance, Jaccard distance, Haversine distance, Sørensen-Dice distance, Manhattan distance, Minkowski distance, Hamming distance, Mahalnobis distance, or another type of distance metric.

7. The method of claim 1, further comprising:
generating a new landmark for the current state if the closest distance to a center of a landmark associated with the previous state is greater than or equal to a predetermined distance; and
updating a counter of a previous landmark associated with the previous state if a center of the previous landmark is the closest distance from the current state and the closest distance is less than the predetermined distance.

8. The method of claim 1, wherein generating the intrinsic reward associated with the exploration by the agent is based on the closest distance of the current state of the agent in relation to a landmark associated with a previous state of one or more previous states of the agent in its exploration of the world.

9. The method of claim 1, further comprising:
generating the sequence of actions based on the dynamic model that maximizes the intrinsic reward; and
selecting a first action from the sequence of actions as the control.

10. The method of claim 9, wherein generation of the sequence of actions that maximizes the intrinsic reward comprises:
generating a plurality of sequences, each of the plurality of sequences including an associated number of actions capable of resulting in possible future states of the agent;
generating intrinsic rewards associated with the possible future states in each of the plurality of sequences;
summing the intrinsic rewards to generate a total reward for each of the plurality of sequences; and
selecting one of the plurality of sequences that has a highest total reward as the sequence of actions that maximizes the intrinsic reward.

11. The method of claim 1, further comprising executing the control to perturb the current state of the agent in its exploration of the world.

12. A system to conduct non-episodic state space exploration of a world, the world being a real-world system having one or more dimensions, the system comprising:
a computing device;
a non-transitory memory storing instructions that, when executed by the computing device, cause the computing device to execute operations comprising:
receiving a current state of an agent observed in relation to its interaction with the world;
updating one or more parameters of a dynamic model that approximates the world based on the current state of the agent, wherein the parameters as updated improve approximation of the world;
generating an intrinsic reward associated with the exploration by the agent based on a closest distance of the current state of the agent in relation to a previous state of one or more previous states of the agent in its exploration of the world; and
generating a control from a sequence of actions based on the dynamic model to maximize the intrinsic reward, wherein execution of the control perturbs the current state of the agent in its exploration of the world.

13. The system of claim 12, wherein operations associated with receiving the current state comprise sensing one or more signals in relation to the interaction of the agent with the world using one or more sensors.

14. The system of claim 13, wherein operations associated with receiving the current state further comprise estimating the current state from the one or more signals as sensed.

15. The system of claim 12, wherein the operations further comprise generating the dynamic model.

16. The system of claim 15, wherein operations associated with generating the dynamic model comprise:
providing one or more hyper-parameters that define a structure of the dynamic model; and
initializing the one or more parameters of the dynamic model that provide an initialized approximation of the world for the exploration by the agent.

17. The system of claim 12, wherein the closest distance is one of a Euclidian distance, Euclidian distance squared, L1 distance, L-infinity distance, cosine distance, Chebyshev distance, Jaccard distance, Haversine distance, Sørensen-Dice distance, Manhattan distance, Minkowski distance, Hamming distance, Mahalnobis distance, or another type of distance metric.

18. The system of claim 12, wherein the operations further comprise:
generating a new landmark for the current state if the closest distance to a center of a landmark associated with the previous state is greater than or equal to a predetermined distance; and
updating a counter of a previous landmark associated with the previous state if a center of the previous landmark is the closest distance from the current state and the closest distance is less than the predetermined distance.

19. The system of claim 12, wherein generating the intrinsic reward associated with the exploration by the agent is based on the closest distance of the current state of the agent in relation to a landmark associated with a previous state of one or more previous states of the agent in its exploration of the world.

20. The system of claim 12, wherein the operations further comprise:
generating the sequence of actions based on the dynamic model that maximize the intrinsic reward; and
selecting a first action from the sequence of actions as the control.

21. The system of claim 20, wherein operations associated with generation of the sequence of actions that maximizes the intrinsic reward comprises:
generating a plurality of sequences, each of the plurality of sequences including an associated number of actions capable of resulting in possible future states of the agent;
generating intrinsic rewards associated with the possible future states in each of the plurality of sequences;
summing the intrinsic rewards to generate a total reward for each of the plurality of sequences; and
selecting one of the plurality of sequences that has a highest total reward as the sequence of actions that maximizes the intrinsic reward.

22. The system of claim 12, wherein the operations further comprise executing the control to perturb the current state of the agent in its exploration of the world.
